(19) United States
(12) Patent Application Publication
(10) Pub. No.: US 2023/0381068 A1
(43) Pub. Date: Nov. 30, 2023

(54) PERSONAL CARE COMPOSITIONS

(71) Applicant: Reckitt Benckiser Health Limited, Slough, Berkshire (GB)

(72) Inventors: Tamika CARTY, Montvale, NJ (US); Ashwini DALVI, Montvale, NJ (US); Sarah Frances DE SZALAY, West Milford, NJ (US); Jouyeop LEE, Montvale, NJ (US); Han Choi YEW, Montvale, NJ (US)

(21) Appl. No.: 18/254,976

(22) PCT Filed: Nov. 24, 2021
(86) PCT No.: PCT/GB2021/053055

§ 371 Date: May 30, 2023

Related U.S. Application Data
(60) Provisional application No. 63/119,096, filed on Nov. 30, 2020.

(30) Foreign Application Priority Data
Mar. 22, 2021 (GB) 2103952.4

(51) Int. Cl. A61K 8/02; A61K 8/365; A61K 8/362; A61Q 19/10; A61K 8/44; A61K 8/46

(52) U.S. Cl. CPC A61K 8/0208; A61K 8/365; A61K 8/362; A61Q 19/10; A61K 8/442; A61K 8/463; A61K 2800/30; A61K 2800/5424; A61K 2800/5428

(57) ABSTRACT

Personal care compositions comprising non-sulfonate anionic surfactant, amphoteric surfactant, and at least two carboxylic acids or salts thereof, and wherein the compositions contain no more than 30% of the total wt % of a) and b) of sulfonated and do not contain para-chloro-meta-xylenol in an amount above 0.15% wt or salicylic acid in an amount above 0.25% wt. In one embodiment the compositions comprise citric acid as one of the at least two carboxylic acids or salts thereof. In another embodiment, the compositions comprise at least two carboxylic acids or salts having a different number of carboxylic acid groups, and the compositions comprise a higher % wt of the carboxylic acid having the lowest number of carboxylic acid groups. The compositions of the invention are intended to provide cleansing and antimicrobial benefits yet be well tolerated by skin and hair. Also provided are a dispenser and disposable wipe containing a composition of the invention, the use of a composition of the invention to provide an antimicrobial benefit to skin and/or hair, and, a method for providing an antimicrobial benefit to skin and/or hair by contact with a composition of the invention.

The present invention relates to personal care compositions suitable for personal care applications, particularly for application to skin and/or hair to provide a cleansing and/or an antimicrobial effect. More particularly, the compositions according to the present invention provide a cleansing and antimicrobial effect on the skin and/or hair. The compositions are preferably well tolerated by the skin yet provide an appreciable antimicrobial benefit.

### BACKGROUND

Compositions for topical application are well-known in the personal care field, including for cosmetic and dermatological use, as well as in the pharmaceutical fields. Topical compositions are intended to provide at least one specific benefit upon use.

For example, liquid personal care compositions which are primarily intended to provide a cleansing or an antimicrobial effect to the area of skin being treated are well known in the field of personal care products.

Consumers are very aware of the health benefits of using antimicrobial products to protect them from undesirable bacteria and viruses and this desire is increasing as the awareness of personal hygiene increases. Typically, such antimicrobial products are used to cleanse and sanitize the user in many different situations. Examples include hand washing, and, washing of other body parts e.g. when bathing and showering. In particular, antimicrobial personal care compositions are especially desirable in situations where hygienic practices may be variable, or, a high standard of hygiene is vital (for example nurseries, educational establishments, hospitals, public toilets and commercial kitchens).

The use of personal care compositions which provide an effective antimicrobial and cleansing benefit is therefore important to consumers who seek protection from undesirable bacteria and viruses. This in turn helps to reduce the spread of disease.

### PRIOR ART

Personal care compositions, including those intended for cleansing applications are well known in the literature.

Budhian et al in WO2017/072482 disclose treatment compositions which are to be used to impart an antimicrobial effect to animate and inanimate surfaces being treated. The compositions comprise anionic surfactants and as an antimicrobial constituent at least one of lactic acid, citric acid, tartaric acid, the substituted acids thereof, derivatives thereof or salts thereof.

Bruning et disclose in WO2017/055789 personal wash compositions, particularly female intimate cleansing compositions personal wash compositions. The compositions comprise lactic acid to provide an antimicrobial affect in combination with a surfactant system.

Yuan et al in US 2008/0247960 disclose foaming topical compositions for application to the human body, particularly to the skin, which provide both a cleaning benefit and a durable antimicrobial benefit. The compositions comprise anionic surfactants with salicylic acid and optionally other organic acids including citric acid or lactic acid as an antimicrobial constituent.

Rypkema et al in WO2006/027551 disclose a liquid composition for treatment of the skin and/or hair to provide a cleaning and/or sanitizing effect thereto. The compositions comprise sodium lauryl ether sulphate as surfactant, benzoic acid as a biocidal constituent with sodium lactate and citric acid included as buffers.

WO2015/058942 discloses compositions for liquid cleaning and disinfection which comprise the anionic surfactant SLES and two carboxylic acids, one of which is citric acid. The other is chosen from malonic acid, malic acid or glycolic acid. Comparative examples use citric acid in combination with lactic acid.

Skin or hair care compositions comprising a stabilizing acid, such as lactic acid, are disclosed in EP 1 593 371A1.

WO2020/165566 discloses personal care products, particularly hand sanitizer compositions, which contain anionic sulfate surfactants and citric and/or lactic acid.

De Szalay et al in WO2018/078336 disclose female intimate cleansing compositions comprising lactic acid and an anionic constituent system comprising secondary alkane sulfonate compound(s), an N-acyl sarcosinate compound and an aromatic hydrotrope compound to boost the antimicrobial activity of the lactic acid.

Vermeulen et al in WO2013/101932 disclose antibacterial liquid soap compositions which comprise lactic acid/lactate, surfactants and alcohol.

Liquid cleansing compositions for use on skin and comprising an antibacterial system comprising certain organic acids are disclosed in WO2018/022016.

The use of organic acids for microbiological control is also known in applications outside of personal care applications.

Cornford discloses in WO2013/185074 cleaning and sanitizing compositions which may optionally comprise an anionic surfactant. Other optional ingredients include lactic acid or citric acid. However, no examples with these ingredients are provided.

For example, in WO01/64035, acidic antimicrobial compositions for treating food and food contact surfaces are disclosed. These compositions may comprise anionic surfactant and an organic acid.

WO01/94513 discloses biocidal cleaner compositions which comprise between certain anionic surfactants anionic surfactant and an acid which may be selected from various carboxylic acids.

In WO99/29815 dishwashing compositions comprising a salt of a carboxylic acid for antibacterial activity are disclosed.

Concentrated solutions of acid, which may comprise surfactant, are disclosed for use in the fermentation of hops in WO2015/136366.

### Problems the Invention Seeks to Address

The basic principle of personal washing and of antimicrobial protection (sanitization) is recognized to encompass the need to control, or break, the potential chain of infection from person to person, or, from inanimate object to person. Personal care products for use in personal washing, personal hygiene and personal care applications have been used for many years.

Whilst sanitizing personal care products have been used for many years to sanitize skin there remains a need for such compositions which provide a balance of several desirable characteristics.

As cleansing and sanitizing compositions are often used in situations where frequent sanitization is required, or the user wishes for frequent sanitization, the cumulative effect of this frequent use can be significantly detrimental to the skin. With repeated use of such compositions the treated skin can become irritated and/or dry, sometimes severely. This may lead to the skin become cracked and/or feeling tight or sore. The skin may also become discoloured or appear inflamed. Frequent use of sanitizing personal care compositions on the skin may lead to disruption of the integrity of the skin mantel. This in turn may lead to infection.

Prior art sanitizing compositions, especially those containing ingredients which are not well tolerated by the skin, may irritate and/or dry the skin especially with repeated use. As personal sanitization needs to be repeated frequently in order to maintain sanitization and avoid undesirable levels of microbial growth, it is desirable that personal sanitizing compositions provide for effective sanitization yet are suitable for frequent use as they do not negatively affect the skin and/or hair to which they are applied. Such negative effects may deter use of such sanitizing compositions frequently, or, completely.

Thus, there is a need in the art for cleansing compositions which are well tolerated by the skin (even upon repeated use) and which do not cause unacceptable levels of irritation and/or dryness but which still provide effective levels of cleansing and/or sanitizing, preferably both.

There is also a need for sanitizing compositions which users find convenient to use in a variety of situations. Most consumers find sore, irritated and/or dry skin to be inconvenient and uncomfortable. Thus, the provision of a composition which consumers can use to conveniently and effectively cleanse and sanitize their skin and/or hair, especially on a frequent basis, is desirable.

If cleansing and sanitizing compositions are used on hair, the same considerations regarding dryness apply for the hair. Also, the considerations for the skin which is in contact with the hair being treated are as above.

It is also desirable to provide effective levels of germ kill on both gram-positive bacteria (such as S. aureus) and gram-negative bacteria (such as E. coli) in order to effectively sanitize. To achieve effective sanitization, the compositions should ideally provide at least a 3 log₁₀ reduction of various microorganisms/bacteria when tested according to the standard test protocols of at least one of ASTM E2315-03 “Standard Guide for Assessment of Antimicrobial Activity Using a Time-Kill Procedure on gram-positive and/or gram-negative bacteria or EN 1276: 2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)”. Achieving this level of sanitation is typically difficult without including high levels of alcohol and/or strongly biocidal ingredients in a personal care composition.

Furthermore, it is desirable to provide effective antimicrobial action for personal care compositions in contact times which the user will find convenient.

The present invention seeks to address one or more of the aforementioned technical problems.

In particular, the present invention seeks to provide sanitizing compositions for personal use, which can be effectively used on skin and/or hair to provide cleansing and santization, and which are well tolerated even after repeated use as they do not cause unacceptable levels of detrimental effects to the skin and/or hair such as skin irritation, damage to the skin mantel and/or drying.

It is also an object of the present invention to provide sanitizing compositions for personal use which are convenient to use and/or can be used in a variety of situations to encourage frequent use.

It is also an object of the invention to provide sanitizing compositions for personal use, which can especially be used on skin and/or hair and which are effective on both gram-negative bacteria and/or gram-positive bacteria and a particular object is to provide at least a 3 log₁₀ reduction of various microorganisms/bacteria as detailed herein.

It is also an object of the invention to provide sanitizing compositions for personal use, which can especially be used on skin and/or hair and which exhibit good antimicrobial activity within a convenient contact time for the user, e.g. of up to 60 seconds.

Surprisingly, the inventors have found that one or more of the above problems can be ameliorated by compositions according to the present invention.

In particular, the present inventors have found that effective cleansing and sanitizing compositions for personal use on skin and/or hair, which can even be used on a frequent basis, are obtained when formulated according to the present invention.

The inventors have also found the compositions of the invention are convenient to use. In particular, they do not discourage frequent use as they are well tolerated in personal care applications.

Moreover, the inventors have found that their compositions provide good antimicrobial effects and typically provide at least a 3 log₁₀ reduction of various microorganisms including on both gram-negative and gram-positive bacteria e.g. when tested according to the standard test protocols of at least one of ASTM E2315-03 or EN 1276: 2009.

The inventors have also found their compositions to provide effective cleansing and sanitizing effects with treatment times of up to 60 seconds, e.g. up to 30 seconds.

The compositions of the invention are convenient to use.

The present inventors have found that their compositions do not cause unacceptable skin irritation or damage to the skin mantel. It has also been found that the compositions do not unacceptably dry the skin the skin even after repeated use.

### STATEMENT OF INVENTION

Accordingly, in first embodiment of the first aspect of the invention there are provided personal care compositions comprising:
a) 0.1% wt to 20% wt of at least one non-sulfonate anionic surfactant,
b) at least one amphoteric surfactant,
c) at least two carboxylic acids or salts thereof, and
wherein the at least two carboxylic acids or salts thereof comprises citric acid and the compositions contain no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

In a second embodiment of the first aspect of the invention there is provided personal care compositions comprising:
a) at least one non-sulfonate anionic surfactant,
b) at least one amphoteric surfactant,
c) at least two carboxylic acids or salts having different number of carboxylic acid groups, and
and wherein the compositions comprise a higher % wt of the carboxylic acid having the lowest number of carboxylic acid groups, and no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.
   The following preferred features apply to both aspects of the invention unless stated otherwise.

It is further preferred that the compositions of the invention are liquid compositions.

It is further preferred that the compositions of the invention are aqueous compositions.

It is further preferred that the compositions of the invention comprise water in an amount of from 65% wt to 99% wt, based on the total weight of the composition.

It is also preferred that the non-sulfonate anionic surfactant is selected from the group consisting of alkyl sulfates or salts thereof and alkyl ether sulfates or salts thereof.

It is further preferred that the alkyl sulfates or salts thereof are selected from the group consisting of C₈-C₁₈ alkyl sulfates and their salts and C₈-C₁₈ alkyl ether sulfates and their salts.

It is further preferred that the alkyl sulfate comprises lauryl sulfate and the alkyl ether sulfate comprises lauryl ether sulfate.

In the second embodiment of the invention it is preferred that the personal care compositions comprise of from 0.5% wt to 20% wt of the at least one non-sulfonate anionic surfactant. In both embodiments of the first aspect of the invention if is more preferred that the compositions comprise of from 2% wt to 15% wt of the at least one non-sulfonate anionic surfactant based on the total weight of the composition.

It is preferred that the amphoteric surfactant present in the compositions of the invention comprises at least one betaine surfactant represented by the general formula:

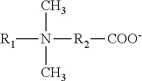

wherein: R₁ is an alkyl group containing from 8 to 18 carbon atoms, or the amido radical which may be represented by the following general formula:

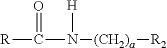

wherein: R is an alkyl group having from 8 to 18 carbon atoms,
a is an integer having a value of from 1 to 4 inclusive, and R₂ is a C₁-C₄ alkylene group.

It is further preferred that the at least one betaine is selected from the group consisting of dodecyl dimethyl betaine, cetyl dimethyl betaine, dodecyl amidopropyldimethyl betaine, tetradecyl dimethyl betaine, tetradecyl amidopropyldimethyl betaine, dodecyl dimethyl ammonium hexanoate and cocoamidopropyl betaine. Cocoamidopropyl betaine is especially preferred according to the present invention.

It is further preferred that the compositions of the invention comprise of from 0.01% wt to 10% wt of the at least one amphoteric surfactant, based on the total weight of the composition.

It is also preferred that the at least two carboxylic acids or salts thereof comprise at least one monocarboxylic acid or salt thereof.

It is further preferred that the least one monocarboxylic acid is selected from the group consisting of acetic acid, glycolic acid, butanoic acid, lactic acid, salicylic acid, benzoic acid and nicotinic acid. It is especially preferred that that at least one monocarboxylic acid comprises lactic acid.

It is further preferred that the compositions of the invention are substantially free of salicylic acid.

According to the second embodiment of the first aspect of the invention it is further preferred that the at least two carboxylic acids or salts thereof comprise at least one tricarboxylic acid or salt thereof. It is also preferred that the at least one tricarboxylic acid is selected from the group consisting of citric acid, propane-1,2,3-tricarboxylic acid, aconitic acid, agaric acid and trimesic acid. It is especially preferred that the at least one tricarboxylic acid comprises citric acid.

According to a both embodiments of the first aspect of the present invention, it is preferred that the at least two carboxylic acids or salts thereof comprise at least one monocarboxylic acid and at least one tricarboxylic acid. An especially preferred combination of at least one monocarboxylic acid and at least one tricarboxylic acid comprises lactic acid and citric acid.

It is further preferred that the at least two carboxylic acids or salts thereof are present in the compositions of the invention in a total amount of from about 0.1% wt to about 10% wt based on the total weight of the composition.

It is further preferred that the compositions of the invention comprise at least one monocarboxylic acid or a salt thereof in an amount of from 0.1% wt. to 5% wt based on the total weight of the composition. It is also preferred that the compositions of the invention comprise at least one tricarboxylic acid or a salt thereof in an amount of from 0.1% wt. to 5% wt based on the total weight of the composition.

According to the first embodiment of the first aspect of the invention it is preferred that the compositions comprise at least one tricarboxylic acid and at least one monocarboxylic acid in a weight ratio in the range of from 5:1 to 1:5, more preferably of from 1:1 to 1:5. According to the second embodiment of the first aspect of the invention it is preferred that the compositions comprise at least one tricarboxylic acid and at least one monocarboxylic acid in a weight ratio in the range of from 1:1.1 to 1:4.

It is further preferred that the compositions of the invention have a pH in the range of from about 3.3 to about 5.

It is further preferred that the compositions of the invention are substantially free of sulfonated compounds.

It is further preferred that the compositions are substantially free of para-chloro-meta-xylenol.

It is further preferred that the compositions of the invention further comprise a nonionic surfactant. It is also further preferred that the compositions nonionic surfactant in amount of from 0.01% wt. to 15% wt based on the total weight of the composition.

It is preferred that the personal care compositions of the invention are formulated to be showering or bathing products, hand washing products, body care products, hand care products, foot care products, facial care products, scalp care products and haircare products.

According to a second aspect of the invention there is provided a dispenser containing a composition according to the invention.

According to a third aspect of the invention, there is provided a disposable wipe comprising a composition of the invention.

According to a fourth aspect of the invention there is provided the use of a composition according to the invention to provide an antimicrobial benefit to skin and/or hair by contacting the composition with skin and/or hair.

According to a fifth aspect of the invention there is provided a method for providing an antimicrobial benefit to skin and/or hair, comprising the steps of contacting a composition according claim 1 with an area of skin and/or hair in a suitable amount and for sufficient time to provide an antimicrobial effect thereon.

The compositions of the invention have been found to address one of more of the aforementioned problems described above.

In particular the inventors have found that by controlling the level of sulphonate compounds, PCMX and salicylic acid and using the carboxylic acids and non-sulfonate surfactants as described herein, personal care compositions providing effective cleansing and sanitization are provided which are well tolerated by skin and hair even upon frequent use.

Other features and advantages of the present invention will be apparent from the following detailed description of the invention and from the accompanying claims.

### DETAILED DESCRIPTION OF THE INVENTION

The compositions according to the present invention will now be described in more detail.

The compositions of the invention are suitably personal care compositions suitable for application to the skin and/or hair of humans, especially to human skin. Preferably the compositions are liquid compositions, more preferably, aqueous compositions and most preferably liquid aqueous compositions.

The term “sulfonated compounds” means any compound containing a sulfonate group.

The term “antimicrobial” as used herein means that the control of at least one of bacteria, fungi and/or virus growth and especially of at least bacteria and viruses.

By the expression “frequent” refers to regular use of the compositions of the invention. The duration between uses will depend on the type of personal care composition. For example, a handwash may be used several times a day or even several times during a couple of hours. A bodywash is more likely to be used daily, or every few days.

The amounts of ingredients stated herein refers to the amount of active ingredient based on the total weight of the composition. Unless otherwise stated, all weights are as wt % based on the total weight of the composition.

It is to be understood that all references to “% wt” amounts are to amounts which are about the stated amount. For example, a reference to ‘of from 5% wt to 10% wt’ is to be construed as ‘of from about 5% wt to about 10% wt’ unless the context requires otherwise.

### Surfactant

The compositions of the invention comprise at least one anionic surfactant which does not contain a sulfonate group, and, at least one amphoteric surfactant.

a) Anionic Surfactant which does not Contain a Sulphonate Group

The compositions of the invention comprise at least one anionic surfactant which does not contain a sulfonate group (hereinafter non-sulfonate anionic surfactant).

It is preferred that the non-sulfonate anionic surfactant is selected from the group consisting of alkyl sulfates or salts thereof and alkyl ether sulfates or salts thereof. These sulfate surfactants provide good foaming characteristics in use and so aid cleansing of the user and also exhibit acceptable skin tolerance characteristics.

One group of preferred non-sulfonate anionic surfactants are alkyl sulfates, especially C₈-C₁₈ alkyl sulfates, most especially C₁₀-C₁₆ alkyl sulfates such as C₁₂-C₁₄ alkyl sulfates and their salts. Any suitable salt may be used and typically an alkali metal (such as sodium, potassium or lithium) or alkaline earth metal salt is used. Most preferably the sodium salt is used. A preferred non-sulfonate anionic surfactant is lauryl sulfate, especially sodium lauryl sulfate known as SLS.

Another group of preferred non-sulfonate anionic surfactants is the alkyl ether sulfates, especially C₈-C₁₈ alkyl ether sulfates, most especially C₁₀-C₁₆ alkyl ether sulfates, such as C₁₂-C₁₄ alkyl ether sulfates and their salts. Again, any suitable salt may be used and typically an alkali metal (such as sodium, potassium or lithium) or alkaline earth metal salt is used. Most preferably the sodium salt is used. An especially preferred non-sulfonate anionic surfactant is lauryl ether sulphate, particularly sodium lauryl ether sulphate known as SLES or sodium laureth sulfate.

Alkyl ether sulfates are preferred according to the present invention as, without wishing to be bound by theory, they are believed to provide good foaming characteristics and skin tolerance in compositions with antimicrobial properties. Combinations of one or more alkyl sulfates with one or more alkyl ether sulfates may be used.

Examples of other non-sulfonate anionic surfactants which may be used according to the present invention include alcohol sulfates, alcohol phosphates and phosphonates, alkyl ester sulfates, sulfate esters of an alkyl phenoxy polyoxyethylene ethanol, alkyl monoglyceride sulfates, alkylaryl sulfates, alkyl carboxylates, alkyl ether carboxylates, alkyl alkoxy carboxylates having 1 to 5 moles of ethylene oxide, alkyl polyglycol ether sulfates (containing up to 10 moles of ethylene oxide), sulfosuccinates, octoxynol or nonoxynol phosphates, taurates, fatty taurides, fatty acid amide polyoxyethylene sulfates, fatty oleyl glycerol sulfates, alkyl phenol ethylene oxide ether sulfates, alkyl phosphates, isethionates, N-acyl taurates, alkyl succinamates and sulfosuccinates, alkyl polysaccharide sulfates, alkyl polyglucoside sulfates, alkyl polyethoxy carboxylates, and sarcosinates or mixtures thereof.

Still further examples of non-sulfonate anionic surfactants which may find use in the compositions of the invention include alkyl-carboxylates, salts (including, for example, sodium, potassium, ammonium, and substituted ammonium salts such as mono-, di-and triethanolamine salts) of soap, C₆-C₂₄ alkyl polyglycol ether sulfates, alkyl ester sulfates such as C₁₄₋₁₆ methyl ester sulfates, fatty oleyl glycerol sulfates, alkyl phenol ethylene oxide ether sulfates, alkyl phosphates, isethionates such as the acyl isethionates, N-acyl taurates, alkyl succinamates, acyl sarcosinates, sulfates of alkyl polysaccharides such as the sulfates of alkyl polyglucoside, branched primary alkyl sulfates, alkyl polyethoxy carboxylates such as those of the formula RO(CH₂CH₂O)ₖCH₂COO⁻M⁺ wherein R is a C₅-C₂₂ alkyl, k is an integer from 0 to 10, and M is a soluble salt-forming cation.

Non-sulfonate anionic compounds which function both as surfactants and as a hydrotrope may be included as either part of the anionic surfactant or as a co-surfactant. Exemplary hydrotropes include C₆-C₁₁ alkyl sulfates and phosphate ester hydrotropes. The hydrotropes are often provided in a salt form with a suitable counterion, such as one or more alkali, or alkali earth metals, such as sodium or potassium, especially sodium. However, other water-soluble cations such as ammonium, mono-, di- and tri-lower alkyl, i.e., C₁₋₄ alkanol ammonium groups can be used in the place of the alkali metal cations. Combinations of two or more non-sulfonate anionic surfactants may also be used in the compositions of the invention if desired. However, according to one aspect of the present invention, it has been found beneficial for the compositions to comprise either an alkyl sulphate or an alkyl ether sulphate as the only anionic surfactant, especially an alkyl ether sulfate. In particular, sodium lauryl sulfate or sodium lauryl ether sulphate are especially preferred as the only non-sulfonate anionic surfactant present in the compositions, most especially sodium lauryl ether sulphate. Especially preferred according to the present invention is the use of an alkyl sulfate and salts thereof and/or an alkyl ether sulphate and salts thereof in the amounts stated herein. Most preferred is the use of lauryl sulfate and/or lauryl ether sulfate, especially the sodium salts thereof in the amounts stated herein.

The personal care compositions of the invention preferably comprise of from 0.1% wt to 20% wt in total of the at least one non-sulfonate anionic surfactant, based on the total weight of the composition, more preferably of from 0.5% wt to 15% wt, especially 1% wt to 12% wt, and most especially 2% wt to 10% wt, e.g. 5% wt to 9% wt, based on the total weight of the composition. According to one embodiment of the invention, the compositions comprise the non-sulfonate anionic surfactant in an amount of from 0.1% wt to 20% wt based on the total weight of the composition. Preferred amounts according to this embodiment are as given above.

Desirably a major proportion of the total amount of non-sulfate anionic surfactant present in the composition of the invention comprises one or more alkyl sulfates and/or alkyl ether sulfates, preferably at least 55% wt., more preferably at least 70% wt., and yet more preferably at least 80% wt., such as at least 90% wt. It is especially preferred that the non-sulfate anionic surfactant constituent comprises at least 99% wt., of one or more alkyl ether sulfates. Most preferably the non-sulfate anionic surfactant consists essentially of one or more alkyl sulfates and/or one or more alkyl ether sulfates.

### b) Amphoteric Surfactants

The compositions of the invention comprise at least one amphoteric surfactant.

Exemplary useful amphoteric surfactants include derivatives of secondary and tertiary amines having aliphatic radicals that are straight chain or branched, and wherein one of the aliphatic substituents contains from about 8 to 18 carbon atoms and at least one of the aliphatic substituents contains an anionic water-solubilizing group, e.g., a carboxy, sulfonate, or a sulfate group. Non-limiting examples of compounds falling within this description include: sodium 3-(dodecylamino)propionate, sodium 3-(dodecylamino)propane-1-sulfonate, sodium 2-(dodecylamino)ethyl sulfate, sodium 2-(dimethylamino)octadecanoate, disodium 3-(N carboxymethyldodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine.

Further exemplary useful amphoteric surfactants include betaines including phosphobetaines. According to one aspect of the invention, betaine surfactants are especially preferred as the amphoteric surfactant.

Exemplary useful betaine surfactants which may be represented by the general formula:

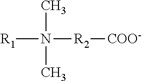

wherein: R₁ is an alkyl group containing from 8 to 18 carbon atoms, or the amido radical which may be represented by the following general formula:

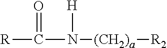

wherein: R is an alkyl group having from 8 to 18 carbon atoms,
a is an integer having a value of from 1 to 4 inclusive, and R₂ is a C₁-C₄ alkylene group.

Examples of preferred betaines according to the present invention include dodecyl dimethyl betaine, cetyl dimethyl betaine, dodecyl amidopropyldimethyl betaine, tetradecyl dimethyl betaine, tetradecyl amidopropyldimethyl betaine, dodecyl dimethyl ammonium hexanoate and cocoamidopropyl betaine. Cocoamidopropyl betaine is an especially preferred betaine according to the present invention.

Mixtures of two or more amphoteric surfactants may be used in the compositions of the invention. If a mixture is used, it is preferred that the mixture comprises at least one betaine.

The at least one amphoteric surfactant, e.g. betaine, may be present in any effective amount, and preferably in amounts of from 0.01% wt. to 10% wt., preferably of from 0.025% wt to 7.5% wt., preferably of from 0.05% wt to 5% wt., such as of from 0.1% wt to 2.5% wt., based on the total weight of the composition.

The compositions of the invention may comprise an equal amount (% wt) of non-sulfonate anionic surfactant and amphoteric surfactant. However, it is preferred that the compositions comprise a greater % wt of non-sulfonate anionic surfactant than amphoteric surfactant. According to one embodiment it has been found that the weight ratio of non-sulfonate anionic surfactant to amphoteric surfactant is in the range of from 10:1 to 60:1, preferably 20 to 55:1, more preferably 30:1 to 50:1, such as 40:1 to 50:1. In this embodiment, nonionic surfactant is not typically present in the compositions. According to another embodiment it has been found that especially good results are obtained with the compositions of the invention when the weight ratio of non-sulfonate anionic surfactant to amphoteric surfactant is in the range of from 2:1 to 10:1, preferably 3:1 to 9:1, more preferably 4:1 to 8:1, such as 5:1 to 7:1. In this embodiment, nonionic surfactant is also typically present in the compositions in the amounts described herein.

### Carboxylic Acids

The compositions of the present invention comprise at least two one carboxylic acids or salts thereof, however the compositions of the invention do not contain salicylic acid in an amount above 0.25% wt. The compositions of the present invention may comprise monocarboxylic acids, dicarboxylic acids and/or tricarboxylic acids and mixtures thereof. Any carboxylic acids suitable for inclusion in personal care compositions may be included in the compositions of the invention. Typically, the carboxylic acids used are C₂-C₂₅ carboxylic acids, preferably C₃-C₂₂. According to one specific embodiment of the invention, the at least two carboxylic acids or salts thereof comprises citric acid. In this embodiment, the compositions may also comprise an additional tricarboxylic acid, preferably one disclosed hereinbelow.

In some embodiments of the invention the acids are preferred to the salts for inclusion in the personal care compositions. Combinations of acids and salts may also be used if desired. The carboxylic acids or salts thereof used in the compositions of the invention are those which exhibit antimicrobial properties yet are suitable for use in personal care formulations.

For the sake of brevity, the references herein to any of the carboxylic acids (generic or specific) includes reference to any salts thereof unless the context requires otherwise.

According to one aspect of the invention, it is especially preferred that the at least two carboxylic acids or salts thereof comprise at least one monocarboxylic acid or salt thereof.

Any monocarboxylic acid suitable for use in personal care compositions may be used according to the invention, including acetic acid, glycolic acid, butanoic acid, lactic acid, salicylic acid, benzoic acid and nicotinic acid. If salicylic acid is used, it is not present in the compositions in an amount above 0.25% wt, preferably not above 0.2% wt, more preferably not above 0.15% wt, most preferably not above 0.1% wt and especially not above 0.05% wt. It is preferred that the compositions of the invention are substantially free from salicylic acid and most especially that they are free of salicylic acid. Lactic acid is especially preferred according to the invention. According to one embodiment of the present invention, the compositions comprise less than 1% wt of salicylic acid, preferably less than 0.5% wt salicylic acid based on the total weight of the compositions, and most preferably are substantially free of salicylic acid and most preferably are free of salicylic acid.

Any dicarboxylic acids suitable for use in personal care compositions may be used in the compositions of the invention including oxalic, acid, succinic acid, malonic acid, tartaric acid, glutaric acid and adipic acid.

It is further preferred that the at least two carboxylic acids or salts thereof comprise at least one tricarboxylic acid or salt thereof. It is also preferred that the at least one tricarboxylic acid is selected from the group consisting of citric acid, propane-1,2,3-tricarboxylic acid, aconitic acid, agaric acid and trimesic acid. It is especially preferred that the at least one tricarboxylic acid comprises citric acid.

According to a particular aspect of the present invention, it is preferred that the at least two carboxylic acids or salts thereof comprise at least one monocarboxylic acid and at least one tricarboxylic acid. An especially preferred combination of at least one monocarboxylic acid and at least one tricarboxylic acid comprises lactic acid and citric acid.

It is also possible, according to the present invention to use at least one dicarboxylic acid with either at least one monocarboxylic acid and/or at least one tricarboxylic acid.

Preferably the at least two carboxylic acids or salts thereof are present in the compositions of the invention in a total amount of from about 0.1% wt. to about 10% wt., more preferably about 0.25% wt. to 7.5% wt., most preferably 0.5% wt. to 5% wt., such as 0.75% wt. to 4.5% wt., especially 1% wt. to 3% wt., e.g. 1.5% wt. to 2.5% wt., based on the total weight of the composition.

It is further preferred that the compositions of the invention comprise at least one monocarboxylic acid or a salt thereof in an amount of from about 0.1% wt. to about 5% wt., more preferably 0.25% wt. to about 4.5% wt., especially more preferably 0.5% wt. to 3.5% wt., most preferably 0.75% wt to 3% wt., such as 0.9% wt to 2.75% wt, e.g. 1% wt. to 2.5% wt. based on the total weight of the composition. It is also preferred that the compositions of the invention comprise at least one tricarboxylic acid or a salt thereof in an amount of from 0.1% wt. to 5% wt., more preferably 0.25% wt. to 4.5% wt., especially more preferably 0.5% wt. to 3.5% wt., most preferably 0.75% wt to 3% wt., such as 0.9% wt to 2.75% wt, e.g. 1% wt. to 2.5% wt. based on the total weight of the composition.

The compositions of the invention may contain the at least one monocarboxylic acid or the at least one tricarboxylic acid in the aforementioned amounts. According to one aspect of the invention, the compositions comprise both the at least one monocarboxylic acid and the at least one tricarboxylic acid in the aforementioned amounts.

If the compositions of the invention comprise dicarboxylic acids, these acids may also be present in the amounts given above for the at least one monocarboxylic acid and the at least one tricarboxylic acid.

Particularly preferred carboxylic acids and mixtures thereof according to the invention, and the preferred amounts thereof, are disclosed in one or more of the Examples.

The inventors have observed that good results are obtained when the compositions of the invention comprise at least one tricarboxylic acid and at least one monocarboxylic acid in a weight ratio in the range of from 5:1 to 1:5, more preferably 2.5:1 to 1:5. The inventors have observed that especially good results are obtained when the compositions of the invention comprise at least one tricarboxylic acid and at least one monocarboxylic acid in an equal wt % and more preferably in a higher wt % of the monocarboxylic acid than of the tricarboxylic acid. Accordingly, the wt ratio of the at least one tricarboxylic acid to the at least one monocarboxylic acid is most preferably in the range of from 1:1 to 1:5, such as 1:1.1 to 1:4, e.g. 1:1.25 to 1:3 or 1:1.3 to 1:2.5 and most especially 1:1.4 to 1:2. In one particular embodiment of the invention the compositions comprise at least one tricarboxylic acid and at least one monocarboxylic acid in a weight ratio of 1:1.5.

Preferably citric acid and lactic acid are present in the aforementioned ratios.

### Sulfonated Compounds

The compositions of the invention comprise no more than 30%, of the total wt % of the at least one non-sulfonate anionic surfactant and the at least one amphoteric surfactant, of sulfonated compounds. It is more preferred that the compositions comprise no more than 25%, most preferably no more than 20%, especially no more than 15%, more especially no more than 10%, such as no more than 5% of the total wt % of the at least one non-sulfonate anionic surfactant and the at least one amphoteric surfactant, of sulfonated compounds. It is especially preferred that the compositions of the invention are substantially free of sulfonated compounds and most especially that the compositions are free of sulfonated compounds.

The inventors have found that by controlling the content of sulfonated compounds in the compositions of the invention, and by including the other components of the invention, effective cleansing compositions with antimicrobial benefits can be provided without undesirable levels of harshness on the skin and/or hair being treated.

Examples of sulfonate containing compounds which may typically be included in personal care compositions include anionic sulfonate surfactants such as alcohol sulfonates, alkyl diphenyl ether sulfonates, alkyl sulfonates such as C₆-C₂₂ primary or secondary alkane sulfonates, C₆-C₂₀ linear alkylbenzene sulfonates, olefin sulfonates, beta-alkoxy alkane sulfonates, alkyl ether sulfonates, ethoxylated alkyl sulfonates, alkylaryl sulfonates, alkyl monoglyceride sulfonates, acyl glycerol sulfonates and paraffin sulfonates, sulfonated polycarboxylic acids, isethionates, N-acyl taurates, and sulfosuccinates and the monoesters and diesters thereof.

Additional anionic compounds containing a sulfonate group and functioning both as a surfactant and as a hydrotrope which may typically be used in personal care compositions include benzene sulfonates, naphthalene sulfonates, C₁-C₁₁ alkyl benzene sulfonates, xylene sulfonates, toluene sulfonates, cumene sulfonates, C₅-C₁₁ alkyl sulfonates and alkyl diphenyloxide disulfonate and their salts.

The amounts of such sulfonated compounds are controlled in the compositions of the invention. Such control can lead, for example, to improved skin and hair tolerance of the compositions of the invention compared to similar compositions containing higher levels of sulfonated compounds.

According to a first embodiment of the first aspect of the invention there is therefore provided personal care compositions comprising:
a) 0.1% wt to 20% wt of at least one non-sulfonate anionic surfactant,
b) at least one amphoteric surfactant,
c) at least two carboxylic acids or salts thereof, and
wherein the at least two carboxylic acids or salts thereof comprises citric acid and the composition contains no more than 30% of the total wt % of a) and b) of sulfonated compounds, and, the compositions do not contain para-chloro-meta-xylenol in an amount above 0.15% wt and do not contain salicylic acid in an amount above 0.25% wt.

An especially preferred composition according to the first embodiment of the first aspect of the invention comprises:
a) 0.1% wt to 20% wt of at least one non-sulfonate anionic surfactant comprising alkyl ether sulfate,
b) at least one amphoteric surfactant comprising a betaine,
c) at least two carboxylic acids or salts thereof, and
wherein the at least two carboxylic acids or salts thereof comprises citric acid and lactic acid and wherein the compositions contain no more than 30% of the total wt % of a) and
b) of sulfonated compounds, and, further wherein the compositions do not contain para-chloro-meta-xylenol in an amount above 0.15% wt and do not contain salicylic acid in an amount above 0.25% wt.

According to a second embodiment of the first aspect of the invention there is therefore provided personal care compositions comprising:
a) at least one non-sulfonate anionic surfactant,
b) at least one amphoteric surfactant,
c) at least two carboxylic acids or salts having different number of carboxylic acid groups, and
wherein the compositions comprise a higher % wt of the carboxylic acid having the lowest number of carboxylic acid groups, and no more than 30% of the total wt % of a) and b) of sulfonated compounds, and, the compositions do not contain para-chloro-meta-xylenol in an amount above 0.15% wt and do not contain salicylic acid in an amount above 0.25% wt.

An especially preferred composition according to the second embodiment of the first aspect of the invention comprises:
a) at least one non-sulfonate anionic surfactant comprising alkyl ether sulfate,
b) at least one amphoteric surfactant comprising a betaine,
c) at least two carboxylic acids or salts having different number of carboxylic acid groups and comprising lactic acid and citric acid, and
and wherein the compositions comprise a higher % wt of the lactic acid, and no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

### Optional Ingredients

The compositions of the invention may include one or more further optional constituents which are used to impart one or more desired esthetic or technical benefits to the compositions. Such optional ingredients include additives and adjuvants which are conventional in the personal care field, such as moisturisers, skin conditioning agents, fragrances, essential oils, colorants, preservatives, further antimicrobially active compounds or materials, foam boosters, humectants, opacifiers, antioxidants, chelating agents, thickener and light stabilizers including UV absorbers. The amounts of these various additives and adjuvants are those conventionally used in the field.

The compositions of the invention are preferably liquid in form. They are preferably aqueous based compositions. Especially preferred are liquid, aqueous based, compositions. Water may form the major component of the compositions of the present invention. The compositions typically comprise water in an amount in the range of from 65% wt to 99% wt, based on the total weight of the composition. Preferably the compositions comprise an amount of from 70% wt to 98% wt water, more preferably of from 75% wt to 95% wt, even more preferably in the range of from 80% wt to 90 wt %.

The water may be tap water but is preferably distilled water and is most preferably deionized water or “soft” water. If the water is tap water, it is preferably substantially free of any undesirable impurities such as organics or inorganics, especially minerals salts which are present in hard water which may undesirably interfere with the operation of the ingredients present in the compositions according to the present invention.

The compositions of the invention may include one or more chelating agents. Exemplary useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. If present, chelating agents are typically included in a total amount in the range of from 0.01 to 0.5% wt., especially of from 0.01 to 0.25% wt based on the total weight of the composition.

The compositions of the invention may comprise one or more humectants, for example polyhydric alcohols including polyalkylene glycols as well as alkylene polyols and their derivatives, such as propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. Glycerine is an especially preferred humectant according to the present invention. One or more humectants may be included in the compositions of the invention. Aloe vera, in one or more of its forms, is preferred as being a naturally derived product. When present, one or more humectants may be included in effective total amounts, advantageously from 0.01-5% wt., preferably from 0.01-3% wt., such as 0.5% wt to 2% wt based on the total weight of the composition of which it/they form(s) a part.

The compositions of the invention may, if desired, include suitable pH adjusting agents including phosphorus containing compounds, monovalent and polyvalent salts such as of silicates, carbonates, and borates, certain acids and bases, tartrates and certain acetates. The use of a base as a pH adjuster, such as a hydroxide, for example an alkali metal or alkaline earth metal hydroxide, is preferred according to the present invention. The use of sodium hydroxide is especially preferred as a pH adjustment agent in the compositions of the invention.

Other pH buffering compounds which may also be used in the compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be desirable to use as buffers such materials as aluminosilicates (zeolites), borates, aluminates and certain organic materials such as gluconates, succinates, maleates, and their alkali metal salts.

When present, the pH adjusting agent is present in an amount effective to adjust or maintain the pH of the inventive composition within a target pH range. Typically, the pH adjusting agent may be included in relatively minor amounts such as from 0.01-5% wt. dependent on the degree of pH adjustment required to achieve the desired pH. If included, they are desirably present in amounts from 0.1-2.5% wt, such as 1-2% wt.

It is preferred that the compositions of the invention comprise at least one preservative. Preservatives are added to inhibit the growth of bacteria, fungi and/or yeasts and many suitable preservatives are known in the art. In the compositions of the invention it is especially preferred that a preservative which is effective against fungi and/or moulds is included therein. Exemplary useful preservatives include benzoates, such as sodium benzoate. Parabens may also be used but are less preferred than benzoates. Advantageously one or more preservatives may be included in amounts of about 0.05% wt. to about 2% by weight, based on the total weight of the composition of which the preservative forms a part. When present, the preservative is included in any amount found to be effective in retarding or inhibiting the growth of undesired microorganisms in the compositions of the invention, particularly during storage for several months at room temperature. The preservative is advantageously present in amounts of up to about 2% wt., preferably are present in amounts of from about 0.001% wt. to about 1.5% wt., and most preferably is present in an amount of from about 0.01% wt. to 1.25% wt, such as from about 0.05% wt. to 1% wt based on the total weight of the composition of which it forms a part.

The compositions of the invention may also include a material which is able to boost the effect of the preservative. These materials are herein termed “preservative boosters”. Such preservative boosters predominantly exhibit other characteristics as well. They typically exhibit some degree of antimicrobial effect although not usually sufficient to be used alone to provide either the preservative effect, or, the antimicrobial effect required for the compositions. Alcohols, such as glycols, may exhibit this preservative booster effect, especially alkyl glycols, e.g. C₂-C₁₀ glycols. One such example is caprylyl glycol which is a preferred ingredient of the compositions of the invention. Caprylyl glycol's main function is to provide emolliency properties to the compositions, but, as it has a degree of antimicrobial activity, it also assists the preservative and provides a boost in preservative power to the compositions. A suitable commercially available caprylyl glycol product is Microcare® CLG (ex Thor). Other suitable preservative boosters include benzyl alcohol. The preservative booster is advantageously present in amounts of up to about 1% wt., preferably are present in amounts of from about 0.001% wt. to about 0.5% wt., and most preferably is present in an amount of from about 0.01% wt. to 0.25% wt, such as from about 0.01% wt. to 0.2% wt based on the total weight of the composition of which it forms a part.

The topical compositions may include a fragrance, which may be based on natural and/or synthetic fragrances and most commonly are mixtures or blends of a plurality of such fragrances, optionally in conjunction with a carrier such as an organic solvent or a mixture of organic solvents in which the fragrances are dissolved, suspended or dispersed. When present the fragrance may be present in any effective amount such that it can be discerned by a user of the composition. However, it is advantageously present in amounts of up to about 1% wt., preferably are present in amounts of from about 0.0001% wt. to about 0.5% wt based on the total weight of the composition of which it forms a part.

The inventive compositions may include one or more colorants, e.g., dyes or pigments useful in personal care compositions to impart a desired color or tint to the compositions. Any colorant which is compatible with the other constituents in the compositions of the invention may be included and in any suitable amount required to achieve the desired visual effect. Advantageously one or more colorants may be added in amounts of about 0.001% wt. to about 0.5% by weight, based on the total weight of the composition of which the colorant(s) forms a part.

If a pearlescent appearance is desired for the compositions of the invention, any suitable pearlizing agents may be added in conventional amounts. A preferred type of pearlizing agent is the glycol stearates, such as ethylene glycol monostearate (EGMS). When present, the pearlizer is typically included in amounts of from about from 0.001 to 2% wt., preferably in amounts from 0.01 to 1.5 wt., e.g. of from about 0.05 to 1% wt. based on the total weight of the composition of which it forms a part.

Another optional ingredient which may be included in the compositions of the invention is an opacifier, e.g. a latex, to provide opacification of the composition. Such are materials which are typically emulsions, dispersions or suspensions of a water insoluble polymer or copolymer in a carrier. Any suitable commercial opacifier may be used if an opaque composition is desired, such as those available under the trademark ACUSOL (ex. Rohm & Haas Inc.). When present in a composition according to the present invention, the opacifier may be present in amounts of up to about 5% wt., preferably are present in amounts of from about 0.001% wt. to about 3% wt., preferably are present in amount from about 0.1% wt. to about 1.2% wt, and most preferably are present in amounts of from about 0.1% wt. to about 1% wt., based on the total weight of the composition of which it forms a part.

The compositions of the invention may comprise one or more nonionic surfactants. According to one embodiment of the invention it is especially preferred to include a nonionic surfactant in the compositions of the invention. Suitable nonionic surfactants include ethoxylated alkylphenols, ethoxylated and propoxylated fatty alcohols, polyethylene glycol ethers of methyl glucose, polyethylene glycol ethers of sorbitol, ethylene oxide, propylene oxide block copolymers, ethoxylated esters of fatty (C₆-C₂₄) acids, condensation products of ethylene oxide with long chain amines or amides, and mixtures thereof. Further exemplary nonionic surfactants include, but are not limited to: methyl gluceth-10, PEG-20 methyl glucose distearate, PEG-20 methyl glucose sesquistearate, C₁₁-C₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethyiene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene 80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof. Alkyl polyglucosides are an especially preferred type of nonionic surfactants according to the present invention. Any suitable alkyl polyglucoside may be used. However, C₆-C₁₈ alkyl glucosides are especially preferred, for example C₈-C₁₆ alkyl glucosides such as C₁₀ glucoside. This C₁₀ glucoside is available as Planteren™ 2000 ex Cognis. Other nonionic surfactants, although not specifically disclosed herein but known to the art may also be used.

If present, the nonionic surfactant(s) is/are preferably included in a total amount of from about 0.01% wt. to about 15% wt., preferably of from 0.1% wt. to 10% wt., preferably 0.5% wt to 7.5% wt., more preferably 1% wt to 5% wt and especially 1.5% wt to 3% wt., based on the total weight of the composition of which they form a part.

The inventors have also found personal care compositions comprising nonionic surfactants as below to be surprisingly beneficial. The personal care compositions comprise:
a) at least one non-sulfonate anionic surfactant,
b) at least one amphoteric surfactant,
c) at least two carboxylic acids or salts,
d) at least one nonionic surfactant, and
wherein the compositions comprise no more than 30% of the total wt % of a) and b) of sulfonated compounds.

A foam booster (which improves the foaming characteristics of the surfactant(s), especially anionic surfactant, present in the compositions of the inventions) may also be included. Such foam boosters are typically nonionic surfactants. Preferred foam boosters are based on one or more alkanolamides which provide composition thickening, foam enhancement, and foam stability. Exemplary alkanolamides which provide such a foam boosting function include but are not limited to: cocamide MEA (monoethamolamine), cocamide DEA (diethamolamine), soyamide DEA, lauramide DEA, oleamide MIPA (monoisopropanolamide), stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. When present, the one or more alkanolamides are present in amounts of up to about 10% wt., but, are preferably included in amounts of from about 0.1-10% wt, such as 0.2-5% wt, for example 0.5-2.5% wt based on the total weight of the topical composition of which they form a part.

Where the compositions require a viscosity modifier, any suitable modifier in an appropriate amount to achieve the desired degree of viscosity modification may be used. For example, sodium chloride may be used to thicken the compositions of the invention. Typically, the sodium chloride is added in the form of a solution in a concentration and an amount suitable to achieve the desired degree of thickening. Typically, the viscosity adjuster, if present, is used in amounts of 1% wt or less, preferably in the range of from 0.01% wt to 0.75% wt, more preferably 0.05% wt to 0.5% wt, such as 0.1% wt to 0.4% wt.

The compositions of the invention may optionally comprise further antimicrobially active materials which are effective against gram-negative and/or gram-positive bacteria provided they are compatible with the other constituents present in the composition. Exemplary useful materials which may be used as the further antimicrobially active material include one or more of antimicrobial agents including: pyrithiones, (especially zinc pyrithione which is also known as ZPT), dimethyldimethylol hydantoin (Glydant®), methylchloroisothiazolinone/methylisothiazolinone (Kathon CG®), sodium sulfite, sodium bisulfite, zinc sulfate, imidazolidinyl urea (Germall 115@), diazolidinyl urea (Germaill II®), benzyl alcohol, 2-bromo-2-nitropropane-1,3-diol (Bronopol®), formalin (formaldehyde), iodopropenyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal dichlorobenzyl alcohol, captan, chlorphenenesin, dichlorophene, chlorbutanol, glyceryl laurate, halogenated diphenyl ethers for example 2,4,4′-trichloro-2′-hydroxy-diphenyl ether (Triclosan® or TCS), phenolic compounds like phenol and aromatic halophenols, 2-hydroxydiphenylmethane, resorcinol and its derivatives, bisphenolic compounds, benzoic esters (parabens) and halogenated carbanilides. Para-chloro-meta-xylenol (PCMX) may optionally be used in the compositions of the invention according to some aspects of the invention, to provide an antimicrobial effect in addition to the carboxylic acids and the amphoteric surfactants. The compositions of the invention do not contain para-chloro-meta-xylenol in an amount of above 0.15% wt. If used, PCMX is present in an amount of at most 0.15% wt, preferably less than 0.1% wt, more preferably less than 0.05% wt based on the total weight of the compositions. The compositions of the invention are preferably substantially free of PCMX and most preferably free of PCMX.

The one or more one further antimicrobially active materials may be present in conventional amounts typically of from about from 0.001-3% wt., preferably in amounts from 0.1-2% wt., but are most desirably present from about 0.1-0.5% wt. based on the total weight of the composition of which they form a part.

The compositions of the invention may include one or more cationic Polyquaternium-type polymers if it is desired to impart moisturizing or conditioning properties to the compositions of the invention and if compatible with the surfactant system used. Such materials may also have mild antimicrobial effects dependent upon the Polyquaternium polymer used. Such materials are well known to the art of topical compositions.

The one or more cationic Polyquaternium-type polymers may be present in amounts of from about from 0.001 to 2.5% wt., preferably in amounts from 0.01 to 2% wt., but are most desirably present in weight percentages from about 0.05 to 1% wt. based on the total weight of the composition of which they form a part.

The compositions may include one or more antioxidant constituents if desired. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids, tocopherols e.g., tocopherol acetate, tocotrienols, and ubiquinone. Natural extracts containing antioxidants suitable for use in the topical compositions of this invention, include but not limited to, extracts containing flavonoids and isoflavonoids and their derivatives, extracts containing resveratrol and the like. Examples of such natural extracts include grape seed, green tea, pine bark and propolis. When present the total amount of such antioxidants are usually not in excess of 5% wt, preferably are present in amounts of from 0.0001-4% wt. based on the total weight of the composition of which it forms a part.

The compositions may comprise a thickener, if required, to achieve the desired viscosity for the compositions. Typically a polysaccharide based thickener may be included e.g., cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, and derivatives thereof including methyl cellulose ethyl cellulose, hydroxymethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxymethyl cellulose and ethyl hydroxy ethyl cellulose. Also suitable are naturally occurring polysaccharide polymers such as xanthan gum, guar gum, locust bean gum, tragacanth gum, or derivatives thereof, polycarboxylate polymers, polyacrylamides, clays, and mixtures thereof.

The polysaccharide-based thickener constituent, particularly the cellulose based thickener constituent, may be present in any effective amount, and are preferably present in amounts of from 0.01% wt. to 7.5% wt. based on the total weight of the composition.

The compositions of the invention may include one or more essential oils which are selected to provide a so-called “aromatherapy benefit” for the user. Essential oils are well known in personal care products. The compositions of the invention may comprise one or more essential oils in conventional amounts. It is also to be recognized that when used, an essential oil or oils providing an aromatherapy benefit may replace all or part of any further fragrance constituent in the compositions of the invention as many of the essential oils providing an aromatherapy benefit are odiferous. These one or more essential oils may be used in the compositions of the invention in an amount of about 0.00001 wt. % to about 1 wt. %, based on the total weight of the composition.

The compositions may include one or more light stabilizers as well as UV absorbers. Such materials are known to be useful in personal care compositions and impart a degree of stability to the compositions which comprise one or more components which are typically deleteriously affected when exposed to certain sources of light, e.g., sunlight, fluorescent light sources. Other such materials are known to stabilize or improve the effect of colorants which may be present in the compositions. Any cosmetically acceptable material or compound which provides protection for one or more of the constituents in the inventive compositions from photolytic degradation or photo-oxidative degradation may be used. When present, the one or more light stabilizers as well as UV absorbers may be included in any effective amount; advantageously such materials are present in amounts of from 0.0001-1% wt., preferably from 0.001-0.25% wt. based on the total weight of the composition of which it forms a part.

Plant or flower extracts, oils or essences may also be included in the compositions of the invention, to provide desirable benefits such as additional antimicrobial properties (for example tea tree oil), moisturizing properties, soothing properties, conditioning properties and/or fragrance. Suitable extracts are well known to the formulator of personal care products. If present such extracts are included in conventional amounts and are usually not in excess of 2% wt, preferably are present in amounts of from 0.0001-1.5% wt, such as 0.01 to 1% wt, based on the total weight of the topical composition of which they form a part.

The compositions may comprise one or more aliphatic alcohols, such as ethanol or propanol (including propan-1-ol and propan-2-ol) if desired. If present, the alcohol is preferably present in an amount of 0.1 wt % to 20 wt %, more preferably 0.5% wt % to 10% wt %, most preferably 1 wt % to 5 wt %. In other embodiments of the invention it is preferred that the compositions comprise less than 5 wt % total alcohol, preferably less than 2.5 wt % and most preferably that they are substantially free of alcohol and especially that they are free of alcohol. The inclusion of high level of alcohols typically renders the compositions harsher to the human skin and hair meaning they are less well tolerated. Additionally, compositions comprising high levels of alcohol may require ‘flammable’ warning information on their packaging. Both these aspects are undesirable and the invention therefore seeks to provide compositions which, if they contain alcohol, do so at levels which do not undesirably damage human skin and/or hair with frequent use. Also, the invention seeks to provide personal care compositions which do not need to carry ‘flammable’ warnings.

In some compositions according to the present invention it may be possible to include cationic surfactants. However, they are usually omitted to avoid the undesirable formation of complexes with the anionic surfactants present. It is especially preferred that the compositions of the invention are substantially free from cationic surfactants and especially that they are free from cationic surfactants.

### pH of the Compositions

The inventors have also found that excellent antimicrobial efficacy and good human skin and/or hair tolerance is provided when the compositions of the invention are formulated to be within a specific acidic pH range of from about 3.3 to about 5 and this range is preferred according to the invention. Preferably the compositions have a pH in the range of from about 3.5 to about 4.8, more preferably of from about 3.75 to about 4.7, even more preferably of from about 3.9 to about 4.6, most preferably of from about 4.0 to about 4.4. It is necessary to balance antimicrobial activity and skin tolerance. Hence very low pH's (especially below pH 3) are avoided according to the invention as they are believed to be more likely to adversely affect the skin/hair potentially leading to skin irritation, sensitization, dryness and/or damage. To control the pH of the compositions, it may be necessary to add a pH adjusting agent to bring it within the range preferred t Immediately upon preparation the compositions of the invention are typically acidic in nature and may require an alkaline pH adjusting agent to bring the pH into the desired range. Suitable pH adjusting agents are discussed hereinbelow.

The pH of the compositions of the invention are those measured at 20° C.

When it is desirable to adjust the pH of the compositions of the invention (e.g. to provide a pH which is better tolerated by skin/air than the starting pH of the composition prior to pH adjustment), one or more pH adjusting agents and/or one or more pH buffers may be included in the compositions in effective amounts to provide the desired pH as described hereinbelow.

### Format of the Compositions of the Invention

The personal care compositions of the invention are preferably liquids. Preferably the compositions are clear, transparent or translucent liquid compositions. If it desired to make the composition opaque or pearlized in appearance a suitable opacifier or pearliser may be added. If a colored composition is desired one or more suitable colorants may be added. Suitable such ingredients are described hereinabove.

The personal care compositions of the invention are intended to provide antimicrobial benefits upon use and thus will be formulated in a physical form suitable for this purpose.

Liquid compositions of the invention can, if desired, be formulated to be in the form of an aqueous solution, a lotion, a cream, an emulsion (including a microemulsion), a mousse or a gel as required. Solid compositions can also be provided according the present invention, for example a bar soap.

It is preferred that the compositions of the inventions produce foam when used. Foam present during use is often perceived by the user as a visual and/or tactile cue of the efficacy/activity of a composition. It also provides the advantage for the user that the coverage of the composition over the area to be treated can be visually assessed which aids the effectiveness of sanitization and reduces the likelihood of areas not being treated.

The liquid compositions of the invention may exhibit a viscosity around that of water (8.9×10⁻⁴ Pa·s at about 25° C.) and for some applications this is preferred. It is also possible to formulate the compositions to have a higher viscosity (e.g. by the inclusion of thickeners) as desired. For some embodiments of the invention, personal care compositions having a viscosity in the range of from 3,000 to 15,000 Centipoise (cP) at 20° C. are preferred, more preferably in the range of from 3,500 to 13,000 cP, even more preferably of from 4,000 to 12,500 cP. The viscosity may be in the range of from 3,000 to 9,000 cP, preferably 3,500 to 8,500 cP, more preferably 4,000 to 8,000 cP, most preferably 4,500 to 7,500 cP e.g. 4,750 to 7,000 cP. Such viscosities have been found to be especially useful as liquid handwash compositions. The viscosity may alternatively be in the range of from 7,000 to 13,000 cP, preferably 7,500 to 12,500 cP, more preferably 8,000 to 12,000 cP, most preferably 8,500 to 11,500 cP e.g. 9,000 to 11,000 cP. Such viscosities have been found to be especially useful as liquid bodywash compositions.

When the compositions of the invention are formulated as liquid personal care products, they may be utilized in a variety of applications, for example as body washes, bathing products such as bath additives, intimate wash products, facial cleansers, liquid hand soaps, foot washes, scalp treatments or shampoos, lotions or creams for the face, hands, feet or body. Solid personal care products, such as bar soaps, cleansing bars and solid hair treatment compositions are also envisaged by the present invention. The compositions of the invention are not intended as treatment agents for the eyes or buccal cavity.

The compositions of the invention may be provided in the form of a concentrated composition which is diluted prior to use to form a composition according to the invention. This provides environmental benefits as it avoids the unnecessary transportation of dilute compositions.

The compositions of the invention are intended for application to the skin and/or hair to provide an antimicrobial benefit thereto. Thus, typically the compositions are provided in a dispenser, or held in, or on, a substrate (such as a non-woven material) for use in such a treatment.

The composition can be packaged in any suitable dispenser to suit its viscosity and intended use by the consumer. Suitable dispensers for the compositions of the invention include bottles, spray dispensers, pump dispensers, aerosol containers, non-deformable dispensers and squeezable dispensers (where pressure is applied to the body of the dispenser, typically manual pressure) to achieve the egress of the composition from the dispenser. Suitable such dispensers are well known in the art.

Suitable substrates in, or on, which the compositions of the invention may be held include wipes which are impregnated (or otherwise carry) a composition of the invention. These wipes may be intended for either single or multiple use and formulated accordingly. Such wipes may be produced from non-woven substrate materials which are well known in the art and include those based on viscose, cotton, cellulose or cellulose derived materials. These items are typically referred to as ‘disposable wipes’. The substrate material is typically soaked in, impregnated with or sprayed with the composition to be delivered by use of the wipe. Such wipes can be easily carried by the user for use outside of the home when required to allow for cleansing when required.

Thus, a second embodiment of the present invention provides a dispenser, containing a composition according to the present invention.

Thus, a third embodiment of the invention provides a disposable wipe comprising a composition of the invention.

### Use of the Compositions of the Invention

Topical application of the compositions of the invention may be performed by contacting the compositions with the skin on any part of the human body, including on the face, neck, chest, back, arms, axilla, hands, legs, feet and scalp. It may also be applied to hair if it is desired to impart an antimicrobial effect to hair. However, primarily, the topical compositions of the invention are intended for application to the skin.

In one aspect of the invention, use of the compositions of the invention provides a rapid, effective and convenient method of providing beneficial cleansing and/or antimicrobial effects to the area being treated whilst being well tolerated by skin and hair. Where appropriate, the compositions of the invention are intended to be washed off with water after use e.g. when formulated as a showering or bathing product, or as another type of washing product.

To use a composition of the invention, the user may simply dispense the desired quantity of the compositions onto the part of the body to be cleansed (e.g. the hands) and rub the composition around that body part. Alternatively, the composition may be applied to a substrate and the substrate contacted with the user's skin and/or hair. Typically, the compositions of the invention are rinsed off after use.

Contact times between the compositions of the invention and the area of skin or hair to be treated to allow for optimal antimicrobial effects is preferably at least 10 seconds, more preferably at least 15 seconds.

It is preferred that the personal care compositions of the invention are formulated to be showering or bathing products (such as body washes, shower gels and products to be added to bath water), hand washing products, body care products, hand care products, foot care products, facial care products, scalp care products or haircare products.

Thus, in a fourth embodiment, the present invention provides the use of compositions according to the present invention to provide an antimicrobial benefit to skin and/or hair by contacting the composition with skin and/or hair.

Thus, in a fifth embodiment, the present invention provides a method for providing an antimicrobial benefit to skin and/or hair, comprising the steps of contacting compositions according to the present invention with an area of skin and/or hair in a suitable amount and for sufficient time to provide an antimicrobial effect thereon.

### Antimicrobial Activity of the Compositions

The compositions of the present invention preferably provide effective antimicrobial activity (germ killing properties) upon both gram-positive bacteria, such as Staphylococcus aureus (S. aureus), and gram-negative bacteria, such as Escherichia co/i (E. coli). The antimicrobial action of the compositions of the invention has been tested using the specified bacterial strains shown in the Examples, but other suitable strains could also be employed to demonstrate the antimicrobial effectiveness of the compositions of the invention.

The inventors have observed that tricarboxylic acids, especially citric acid, are very effective upon gram-negative bacteria (such as E. coli), but, slightly less effective against gram-positive bacteria (such as S. aureus). The inventors have also observed that monocarboxylic acids, especially lactic acid, are effective against gram-positive bacteria (such as S. aureus) and help to provide a good level of antimicrobial activity for the composition against both gram-negative bacteria and gram-positive bacteria when used with a tricarboxylic acid.

For the antimicrobial testing results given in the Examples, while there are no established criteria for an absolute “pass” or “fail” result in such tests, it was considered by the inventors of the present compositions that a log₁₀ reduction result of less than 3 constituted a “fail” score, and a log₁₀ reduction result of 3 or more constituted a “pass” score and demonstrated superior performance against the stated gram-positive and gram-negative bacteria. In particular, the inventors found that compositions which achieved scores of 3.5 or more, especially 4 or more, most especially 4.5 or more were much preferred as they exhibited greater antimicrobial effects as the score increased. The most preferred compositions of the invention achieved a log₁₀ reduction score of 5 or more.

The compositions of the invention are also believed to be effective against spores and certain viruses, including enveloped DNA and RNA viruses, such as those which cause the common cold, influenza viruses and coronaviruses.

### Preparation of the Compositions

The compositions of the invention may be produced by any suitable method. One suitable method which may be used to produce the compositions is given below and was used to produce the examples. For any types of ingredients not included in the compositions of the invention, the addition step for ingredient would simply be omitted. The preparation is typically carried out in a suitably sized mixing tank, equipped with a stirrer. Other suitable methods of preparation will be apparent to the person skilled in the art.
1. Add approximately 40% of the total amount of distilled water in the composition at ambient temperature and stir at a sufficient speed to produce a vortex.
2. Heat the water to about 65-70° C.
3. If using a chelating agent, such as tetrasodium EDTA, add and mix until dissolved.
4. Add the one or more tricarboxylic acid(s), such as citric acid (may be added as a solution if desired) and mix until dissolved.
5. Add the non-sulfonate anionic surfactant, such as sodium lauryl ether sulphate, and mix until fully dissolved. (e.g. for about 15 minutes).
6. Add the amphoteric surfactant, such as cocoamidopropyl betaine, and mix until dissolved, maintaining a temperature not above 70° C. A sample may be taken from the mixing tank to check for undissolved non-sulfonate anionic surfactant and/or amphoteric surfactant. If any is found, continue mixing until none is found.
7. If using a preservative system aid, such as caprylyl glycol, add the required amount (pre-heated to a temperature of approximately 35° C. to 40° C. if required to aid addition) and mix until fully dispersed.
8. If using a pearlizer, such as ethylene glycol monostearate, add to the mixture and mix until dissolved, ideally maintaining the temperature of the mixture in the range of about to about 65-70° C. Once the pearlizer has dissolved stop heating.
9. Add approximately 30% of the total amount of distilled water in the composition to the mixture and continue mixing until the mixture is well mixed.
10. If using a humectant, such as glycerin, add as the mixture is cooling (e.g. when the mixture is in the temperature of about 50-55° C.) and mix until dissolved.
11. Add the one or more monocarboxylic acid(s), such as lactic acid (may be added as a solution if desired) and mix until dissolved.
12. Cool the mixture to a temperature of between ambient and about 40° C., e.g. 35-40° C.
13. Add the required amount of preservative (e.g. sodium benzoate) and mix until fully dissolved.
14. If dye is used, separately dissolve it in a sufficient amount of distilled water and add the dye/water mixture to the mixing tank, mix until dissolved.
15. If fragrance is used, add the required amount and mix until dissolved.
16. Measure the pH of the composition (typically will be around pH 3-4) and adjust to a pH within the target range for the composition, typically by using a suitable base (e.g. a 30% wt solution of sodium hydroxide), noting the amount used to achieve the desired pH. Continue mixing the composition until well mixed.
17. Measure the viscosity of the composition and adjust as required. If desired, the viscosity can be adjusted by the addition of a viscosity adjuster such as sodium chloride (which may be used as a solution) noting the amount used to achieve the desired viscosity. Continue mixing until well mixed.
18. Add an amount of distilled water sufficient to make the composition up to 100 parts (water to 100%, or q.s.).
   N.B. The raw material vessels, especially those containing the non-sulfonate anionic surfactant, amphoteric surfactant, carboxylic acids and any perfume may desirably be rinsed with around 1% wt of the total amount of distilled water once the raw materials have been added to the mixing vessel and the rinse water added to the mixing tank.

The above method of preparation may be altered according to the ingredients included in the compositions of the invention. In particular, the order of addition of the optional ingredients may be varied as desired and appropriate. The modifications which may be used include;
Adding the distilled water as a single addition in step 1 so step 9 is omitted.
The at least one monocarboxylic acid, and/or the at least one tricarboxylic acid may be added after the cooling step (step 12).

If the compositions of the invention comprise a foam booster, such as an alkanolamide e.g. cocamide MEA and/or a non-ionic surfactant, such as a glucoside, e.g. decyl glucoside, these are typically added prior to the cooling phase such as after the addition of the amphoteric surfactant.

If the compositions of the invention are formulated as solid compositions, any suitable method of preparation may be used.

The following examples below illustrate exemplary formulations as well as preferred embodiments of the invention. It is to be understood that these examples are provided by way of illustration only and that further useful formulations falling within the scope of the present invention and the claims may be readily produced by one skilled in the art without deviating from the scope and spirit of the invention.

### Examples

Compositions according to the invention are provided in the following Examples. The compositions are all intended for application to human skin/hair. They find particular application as cleansers or treatment compositions for human skin and hair. These compositions were prepared following the method of preparation stated hereinabove.

In the following compositions, the ingredients were used “as supplied” from their respective suppliers and some may contain less than 100% wt. of the active material. The ‘active’ amount of each ingredient is indicated in the following tables.

The percentages given in the tables are as % wt. based on the total weight of the compositions. In each of the compositions deionized water was included in “quantum sufficient” (q.s.) to provide 100 parts by weight of the specific composition as stated in the tables.

For each composition in Table 1, the amount of sodium hydroxide solution given is the amount added to bring the composition to its final desired pH.

The compositions above in the tables below 1 were evaluated for their antimicrobial efficacy against Staphylococcus aureus (ATCC 6538) and Escherichia co/i (ATCC 10536).

The test method used to evaluate the antimicrobial effectiveness of the Examples was an antimicrobial suspension test based on British standard reference No: EN 1276: 2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)” with the following modifications made to the method;
eliminating the interfering substance indicated by the protocol as “dirty or clean conditions” and
increasing the test temperature from 20±1° C. to 37±1° C. (as 37° C. is closer to the average human body temperature than 20° C.).

The test protocol tests the efficacy of the compositions of the present invention and the comparative example against gram-negative and gram-positive bacteria. The test organisms used were Staphylococcus aureus ATCC 6538 and Escherichia coli ATCC 10536.

The bacterial strains were cultured on tryptic soy agar (TSA) slant from frozen stock and incubated for 24 hours. Following incubation, 2^{nd} and 3^{rd} generation transfers were prepared and used to prepare test suspensions as described in the EN 1276:2009 test method. The cell suspensions were adjusted to produce approximately 1.5-5.0×10⁸ CFU mL⁻¹. The growth medium and temperature used were Tryptic soy Agar (TSA) and 35±1° C. Test solutions and test cultures are equilibrated to a test temperature of 37±1° C. in a water bath.

Experimentally, a 1.0 mL of the test culture was exposed to 9.0 mL of the test product for a 1-minute contact time and neutralized in a verified neutralizer. After a 5-minute neutralization time, the neutralized sample was serially diluted, plated on TSA and incubated at 35±1° C. for 48 hours. The average Log₁₀ CFU/mL for the test suspension was calculated and used to compute the log reduction post-treatment. A three log₁₀ reduction of all tested bacterial strains in a 1-minute contact time was chosen to indicate that the tested formulation has the desired level of antimicrobial properties against tested organism(s).

The formula applied to calculate the log reductions was: Log Nc−Log Nd where:

Nc=Number of cfu/ml of the test suspension count and Nd=Number of cfu/ml of the sample count after treatment.

The amounts in the compositions below are given as the % of the active ingredient in the composition unless otherwise stated. The amount of raw material added will therefore be higher than the stated amount for all ingredients which are not 100% active. The amount of raw material added is easily calculated from the information given in the ingredients table below.

TABLE 1
liquid handwash compositions (examples 1 to 6)

| | Ex.1 | Ex.2 | Ex.3 | Ex.4 | Ex.5 | Ex.6 |
|---|---|---|---|---|---|---|
| | % wt | % wt | % wt | % wt | % wt | % wt |
| | active | active | active | active | active | active |
| D.I. water | q.s. | q.s. | q.s. | q.s. | q.s. | q.s. |
| Tetrasodium | 0.087 | 0.087 | 0.087 | 0.087 | 0.087 | 0.087 |
| EDTA | | | | | | |
| Sodium lauryl | 8.4 | 7.0 | 8.4 | 7.0 | 7.0 | 7.0 |
| ether | | | | | | |
| sulfate 2EO | | | | | | |
| (SLES) | | | | | | |
| Cocamide MEA | 0.86 | 0.86 | 0.86 | 0.86 | — | — |
| Cocoamidopropyl | 1.29 | 1.29 | 1.29 | 1.29 | 1.5 | 1.5 |
| betaine | | | | | | |
| Alkyl olefin | — | 0.69 | — | — | — | — |
| sulphonate | | | | | | |
| Lactic acid | 2.15 | 2.15 | 2.0 | 2.5 | 1.5 | 1.5 |
| Citric acid | 0.5 | 0.5 | 0.5 | 0.5 | 1.0 | 1.0 |
| anhydrous | | | | | | |
| Glycerine | 2.0 | 2.0 | 2.0 | 2.0 | 2.0 | 2.0 |
| Sodium chloride | 1.25 | 1.25 | — | — | 0.59 | 0.14 |
| Sodium | 0.47 | 0.47 | — | — | 0.70 | 0.82 |
| hydroxide | | | | | | |
| Sodium benzoate | 0.1 | 0.1 | 0.1 | 0.1 | 0.2 | 0.2 |
| Fragrance | 0.5 | 0.5 | — | — | — | — |
| Ethylene glycol | 2.0 | 2.0 | — | — | — | — |
| monostearate | | | | | | |
| (EGMS) | | | | | | |
| Caprylyl glycol | — | — | — | — | 0.1 | 0.1 |
| Zinc sulphate | — | — | — | — | — | 0.1 |
| heptahydrate | | | | | | |
| Test results | | | | | | |
| pH (final) | | | 3.49 | 3.49 | 4.21 | 4.35 |
| Viscosity cPs | | | | | | 4209 |
| Log₁₀ reduction | | | >4.87 | >5.46 | 3.35 | 5.44 |
| for | | | | | | |
| E.coli.(clean) | | | | | | |
| Logio reduction | | | >5.06 | | 3.39 | 3.52 |
| for | | | | | | |
| S. Aureus. (clean) | | | | | | |
| Log₁₀ reduction | | | | >5.45 | | |
| for | | | | | | |
| E. Coli.(dirty) | | | | | | |
| Log₁₀ reduction | | | | >4.02 | | |
| for | | | | | | |
| S. Aureus.(dirty) | | | | | | |

The content of the sulphonate compound based on the total wt % of the non-sulphonate anionic surfactant and the amphoteric surfactant is 8.3% wt in Example 2.

TABLE 2
liquid handwash compositions (examples 7,
8) and a body wash composition (example
9). Also comparative examples C10 and C11.

| | Ex. 7 | Ex. 8 | Ex. 9 | Ex. C10 | Ex. C11 |
|---|---|---|---|---|---|
| | % wt | % wt | % wt | % wt | % wt |
| | active | active | active | active | active |
| D.I. water | q.s. | q.s. | q.s. | q.s. | q.s. |
| Tetrasodium EDTA | 0.087 | 0.087 | 0.087 | 0.087 | 0.087 |
| Sodium lauryl ether | 7.0 | 7.0 | 10.0 | 7.0 | 7.0 |
| sulfate 2EO (SLES) | | | | | |
| Cocoamidopropyl | 1.5 | 1.5 | 1.5 | 1.5 | 1.29 |
| betaine | | | | | |
| Alkyl polyglucoside | — | — | 0.5 | — | — |
| Cocamide MEA | — | — | 0.5 | — | 0.86 |
| Lactic acid | 1.5 | 1.5 | 1.5 | 1.5 | 2.5 |
| Citric acid anhydrous | 1.0 | 1.0 | 1.0 | 1.0 | — |
| Glycerine | 2.0 | 2.0 | — | 2.0 | — |
| Sodium chloride | 0.2 | 0.2 | 0.3 | 0.23 | — |
| Sodium hydroxide | 0.54 | 0.54 | 0.5 | 0.8 | — |
| Zinc sulphate | — | — | — | — | 0.1 |
| heptahydrate | | | | | |
| Sodium benzoate | 1.0 | 1.0 | 1.0 | 0.2 | 0.1 |
| Fragrance | 0.5 | 0.5 | 0.43 | 0.3 | — |
| Ethylene glycol | 0.5 | 0.5 | 1.0 | 0.5 | — |
| monostearate (EGMS) | | | | | |
| Caprylyl glycol | 0.1 | — | — | 0.1 | 0.4 |
| Para-chloro-meta- | — | — | — | 0.175 | — |
| xylenol (PCMX) | | | | | |
| Dye | 0.02 | 0.02 | — | 0.1 | — |
| Test results | | | | | |
| pH (final) | 4.32 | | | 4.31 | 3.49 |
| Viscosity cPs | 6299 | | | 7678 | |
| Log₁₀ reduction for | >5.32 | | | | >5.46 |
| E. coli.(clean) | | | | | |
| Log₁₀ reduction for | 3.58 | | | | |
| S. Aureus. (clean) | | | | | |
| Log₁₀ reduction for | | | | | |
| E. Coli.(dirty) | | | | | |
| Log₁₀ reduction for | | | | | 3.26 |
| S. Aureus.(dirty) | | | | | |

Details of ingredients used in the foregoing Examples are given in the table below.

| Constituent | Function | Details |
|---|---|---|
| D.I. water | Solvent | Distilled water |
| Tetrasodium EDTA | Chelating agent | 87% active solution of tetrasodium |
| | | EDTA |
| Sodium lauryl ether sulfate | Surfactant - anionic | 70% wt. active solution. |
| (SLES) | non-sulfonate | |
| Cocoamidopropyl betaine | Surfactant - | 29% wt. active solution. |
| | amphoteric | |
| Alkyl polyglucoside | Surfactant - nonionic | Decyl glucoside, available as Planteren ™ |
| | | 2000 ex Cognis, 48-55% active. Used as |
| | | 51% active in calculations. |
| Cocamide MEA | Foam booster | 86% active raw material of cocamide |
| | | MEA. |
| Alkyl olefin sulphonate | Surfactant | 92% active raw material of Alkyl olefin |
| | | sulphonate. |
| Lactic acid | Antimicrobial agent | A 50% wt solution of lactic acid |
| Citric acid anhydrous | Antimicrobial agent | Citric acid anhydrous, purity at least |
| | | 99.5% |
| Glycerine | Humectant | Glycerine purity of 99.7% |
| Sodium chloride | Thickener | Sodium chloride purity of 98% minimum |
| Sodium hydroxide | pH adjuster | A 30% solution of sodium hydroxide (98- |
| | | 100% wt. active) |
| Sodium benzoate | Preservative against | Used as the powdered form, available |
| | yeasts and fungi | commercially from a variety of suppliers. |
| Fragrance | fragrance | Proprietary composition of its supplier |
| Ethylene glycol | Pearlizer | Available commercially as Galaxy ™ 610 |
| monostearate (EGMS). | | from Galaxy Surfactants Ltd. |
| Microcare ® CLG | Emollient and aids | 100% wt caprylyl glycol (ex Thor) |
| | preservative effect | |
| Zinc sulphate heptahydrate | Antimicrobial active | Used as the powder. |

### Results

The results in Tables 1 and 2 demonstrate that the compositions of the present invention show excellent antimicrobial activity on both S. aureus and E. coli. Comparative example C10 contains 0.175% wt PCMX, above the upper limit of 0.15% wt stipulated in claim 1. The excellent antimicrobial activity of the compositions of the present invention shows that PCMX at this high level is not needed. Comparative example C11 which contains lactic acid but no citric acid shows lower activity against S. aureus (dirty) than Example 4 of the invention which contains both lactic acid and citric acid.

The inventors have also noticed that compositions comprising a greater amount of lactic acid than citric acid in combination with the claimed surfactant system are especially advantageous for antimicrobial effects and skin tolerance.

While the invention is susceptible of various modifications and alternative forms, it is to be understood that specific embodiments thereof have been shown by way of example which are not intended to limit the invention to the particular forms disclosed; on the contrary the intention is to cover all modifications, equivalents and alternatives falling within the scope and spirit of the invention as expressed in the appended claims.

What is claimed is:

1. Personal care compositions comprising:
a) 0.1% wt to 20% wt of at least one non-sulfonate anionic surfactant,
b) 0.01% wt to 10% wt of at least one amphoteric surfactant,
c) at least two carboxylic acids or salts thereof, comprising lactic acid or a salt thereof in an amount of from 0.1% wt to 5% wt and citric acid or a salt thereof in an amount of from 0.1% wt to 5% wt, and
wherein the lactic acid is present in a higher wt % than the citric acid, and wherein the compositions contain no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

2. (canceled)

3. A personal care composition according to claim 1, wherein the composition is an aqueous composition and comprises water in an amount of from 65% wt to 99% wt, based on the total weight of the composition.

4. A personal care composition according to claim 1, wherein the non-sulfonate anionic surfactant is selected from the group consisting of alkyl sulfates or salts thereof and alkyl ether sulfates or salts thereof.

5. A personal care composition according to claim 4, wherein the alkyl sulfates or salts thereof are selected from the group consisting of C₈-C₁₈ alkyl sulfates and their salts and C₈-C₁₈ alkyl ether sulfates and their salts.

6. A personal care composition according to claim 1, wherein the composition comprises of from 0.5% wt to 15% wt of the at least one non-sulfonate anionic surfactant, based on the total weight of the composition.

7. A personal care composition according to claim 1, wherein the amphoteric surfactant is selected from the group consisting of betaine surfactants represented by the general formula: 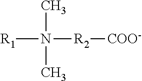
wherein: R₁ is an alkyl group containing from 8 to 18 carbon atoms, or
the amido radical which may be represented by the following general formula: 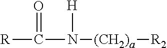
wherein: R is an alkyl group having from 8 to 18 carbon atoms,
a is an integer having a value of from 1 to 4 inclusive, and R₂ is a C₁-C₄ alkylene group.

8-9. (canceled)

10. A personal care composition according to claim 1, wherein the composition is substantially free of at least one of: salicylic acid, sulfonated compounds, para-chloro-meta-xylenol, or combinations thereof.

11. (canceled)

12. A personal care composition according to claim 1, wherein a further tricarboxylic acid is present from the group consisting of propane-1,2,3-tricarboxylic acid, aconitic acid, agaric acid and trimesic acid, and salts thereof.

13. A personal care composition according to claim 1, wherein a further monocarboxylic acid and is present, selected from the group comprising acetic acid, glycolic acid, butanoic acid, benzoic acid and nicotinic acid, or salts thereof.

14-18. (canceled)

19. A personal care composition according to claim 1, wherein the citric acid and lactic acid or salts thereof are present in a weight ratio in the range of from 1:1.1 to 1:4.

20. A personal care composition according to claim 1, wherein the composition has a pH in the range of from about 3.3 to about 5.

21. A personal care composition according to claim 1, wherein the composition further comprises a nonionic surfactant in an amount of from 0.01% wt. to 15% wt.

22. A personal care composition according to claim 1, wherein the personal care composition is selected from the group consisting of showering or bathing products, hand washing products, body care products, hand care products, foot care products, facial care products, scalp care products and haircare products.

23. A dispenser containing a composition according to claim 1.

24. A disposable wipe comprising a composition according to claim 1.

25. A method for providing an antimicrobial benefit to skin and/or hair, comprising the steps of contacting a composition according claim 1 with an area of skin and/or hair in a suitable amount and for sufficient time to provide an antimicrobial effect thereon.

26. A personal care composition according to claim 19, wherein the citric acid and lactic acid or salts thereof are present in a weight ratio in the range of from 1:1.25 to 1:3.

27. A personal care composition according to claim 26, wherein the citric acid and lactic acid or salts thereof are present in a weight ratio in the range of from 1:1.3 to 1:2.5.
